(19) United States
(12) Patent Application Publication
(10) Pub. No.: US 2026/0165657 A1
(43) Pub. Date: Jun. 18, 2026

(54) Synchronous Electromagnetic Power Line Interference Removal Device

(71) Applicant: Cadwell Laboratories, Inc., Kennewick, WA (US)

(72) Inventor: John A. Cadwell, Richland, WA (US)

(21) Appl. No.: 19/421,344

(22) Filed: Dec. 16, 2025

Related U.S. Application Data
(60) Provisional application No. 63/734,622, filed on Dec. 16, 2024.

(51) Int. Cl. A61B 5/00; G06F 18/2413

(52) U.S. Cl. CPC A61B 5/7203; A61B 5/7221; G06F 18/2413; A61B 2560/0214

(57) ABSTRACT

A signal processing apparatus removes the interference due to a mains power supply from a bioelectrical signal reading by using a hardware component to directly measure the mains, generate a digital reference signal using the mains, determine phase and amplitude of the interference, wherein the determining is performed for each segment of segments of a composite signal, measure signal to noise ratio (SNR) for each segment, wherein the mains interference constitutes the signal and the bioelectrical signal constitutes the noise, generate a steady state phase and amplitude of the interference based on the determined phase and amplitude and the SNR, and subtract the steady state interference from the composite signal to obtain the bioelectrical signal.

### CROSS-REFERENCE

The present application relies on U.S. Patent Provisional Application No. 63/734,622, titled “Synchronous Electromagnetic Power Line Interference Removal Device” and filed on Dec. 16, 2024, for priority, which is herein incorporated by reference in its entirety.

### FIELD

The present specification is related generally to the field of signal processing. More specifically, the present specification is related to methods for mitigating electromagnetic interference or electrical noise in both clinical and non-clinical signals.

### BACKGROUND

Electromagnetic interference or electrical noise is a common type of noise that is caused by a power line (also generally called a ‘mains’) and results from a distributed electromagnetic field that is leaking through the local environment and eventually through the power supply. The electrical noise typically causes a sinusoidal harmonic component, ranging from 50 or 60 Hz, plus harmonics, to be added to the sensitive signals of interest and being measured. These signals of interest can include bioelectric signals such as electromyography (EMG), electroencephalography (EEG), electrooculography (EOG), electrocardiogramalectroretinography (ERG) or other types of clinical and non-clinical signals. The interference from the mains is introduced into the bioelectric signals from within the operating environment.

A common method that is widely used to prevent or mitigate electrical noise includes placing sensitive signal generating equipment in shielded rooms. These rooms in which the sensitive devices may be used are shielded with different types of materials that are impermeable to electromagnetic fields, such as copper and iron, among others. Another method is to use the sensitive devices in dedicated spaces that are created at a distance from possible sources of electrical noise. Noise reduction efforts may also include placing the sensitive devices as far as possible from other high noise generating devices. In some cases, a good level of contact impedance, preferably from 1 k to 10 k ohms, is maintained to overcome environmental noise.

When the source of noise is from a mains power supply (hereinafter, the mains power supply has a mains signal and the noise is referred to as power line interference or PLI), a common solution is to use a notch filter which removes most of the unwanted signal (noise) or PLI at its center frequency (either 50 or 60 Hz, depending on the available standard). The noise is removed to avoid data contamination and obtain accurate signals for analysis. In many cases, however, the signal of interest overlaps the mains frequency, such as with EMG measurements. In this case, a notch filter can distort the EMG signal and can introduce damped, sinusoidal ‘ringing’ to the desired EMG signal.

Notch filters, which are also known as band-stop or band-reject filters, have a number of variants. As the name suggests, they are used to eliminate specific frequencies, while allowing all other frequencies. In the context of the present specification, the notch filters that are referred to have a resonant circuit tuned to the frequency from a mains power supply. The resonant response from the notch filter is then subtracted from the signal of a sensitive device.

Notch filters are implemented digitally and applied to time series sampled data. Digital notch filters perform mathematical operations on the time series sampled data to reduce or eliminate the specific set of frequencies that are from a signal. The characteristics of notch filters can be modified by changing the number of poles, the coefficients defining the poles, the frequency or frequencies used, or the filter type, among other parameters. In addition, some algorithms used with notch filters may operate by seeking the signal center frequency and dynamically changing the filter center frequency.

In operation, notch filters extract a power line interference or mains interference signal from a composite bioelectric signal wherein the composite signal includes a true bioelectric signal contaminated with the noise due to the mains signal. If the true bioelectric signal, or portions of it, and the noise signal from the mains are in the same frequency band, then the process of extraction of the mains signal from the composite bioelectric signal for the purpose of allowing only the true bioelectric signal to remain is compromised. Therefore, current implementations of notch filters reduce the effectiveness of the filters and distort the true bioelectric signal.

Abrupt changes in the mains signal can generate large transient artifacts in bioelectric signals, which can perturb a notch resonator. During an EMG exam the needle is moved abruptly followed by a second or two of non-movement while the user listens and looks for physiological effects of the movement. Typically, what is seen is a transient burst due solely to needle movement, followed by a physiological response of the muscle cells that results from the mechanical disturbance. The physiological response, if present, is usually abnormal and indicative of a pathology. The signal seen during this time is referred to as ‘insertional activity’. By way of example, this abrupt needle movement during an EMG needle exam can change the mains signal. The notch resonator takes time to recover after a transient event, and the new power level of the mains may be different than the old level (since the amount of mains interference can change with needle movement), which also adds to the time to settle. While settling, the notch filter artifact is added to the signal instead of being subtracted. This occurs during the several hundred milliseconds that are the most important for observing the insertional activity.

Notch filters require some time in the range of 50 to 200 milliseconds to complete the process of removing the noise frequencies in order to arrive at a true signal. When the mains level changes, the resultant filter output signal includes damped, sinusoidal ‘ringing’ reflective of the perturbation.

Certain aggressive notch strategies, such as a comb filter, add ‘buzz’ or other artifacts to the bio-electrical signal and have long (up to a few seconds) settling times. In EMG and other neurophysiological signals, the physiologic response occurs within a fraction of a second to a few seconds; therefore, the long settling times of the mains signal may not result in an effective removal of interference from the bio-electrical signal (EMG).

In an ideal environment where the amplitude of the mains signal is zero, there is no need for reduction or removal of the mains interference signal component. In such a situation, using a notch filter will distort the bio-electrical signals as the notch filter is unable to remove what is not there. Further, at low to medium amplitudes of the mains signal, picking the mains signal out of the composite signal (the composite signal is a mix of the mains interference signal and the bio-electrical signal) for optimal removal is problematic, and a derived reference is not as stable as desired.

Often, the mains signal has the additional effect of adding harmonics to the bio-electrical signal. By way of example, a third harmonic at 180 Hz may be heard in the form of a buzz in a public address (PA) system where the broadcast audio signal is contaminated by harmonics resulting from power line interference (PLI). Therefore, in addition to removal of PLI, the removal of harmonics due to the mains signal, such as at 180 Hz, is desirable but is not often implemented because the frequency falls in the EMG spectrum and causes the same sorts of artifacts (ringing or buzz) at a higher frequency. Moreover, sometimes higher frequency harmonics are added due to the mains signal. The higher frequency harmonics can be identified and removed if found.

As a result of these limitations, there is no known filtering approach that yields minimal waveform distortions and an acceptable level of ‘ringing’ for many neurophysiology applications. Thus, there is a need for improved methods and systems to enable the accurate recordation of bioelectric signals in compromised environments.

### SUMMARY

The following embodiments and aspects thereof are described and illustrated in conjunction with systems, tools and methods, which are meant to be exemplary and illustrative, and not limiting in scope. The present application discloses numerous embodiments.

In embodiments, the present specification discloses a signal processing apparatus for processing a composite signal comprising a power line interference (PLI) component and a physiological signal of interest (PHSI), wherein the signal processing apparatus comprises: a hardware component configured to measure an analog PLI signal from a source of the PLI component; and a processing system configured to: generate a reference signal by digitizing the measured PLI component; determine a phase and an amplitude of the measured PLI component using the generated reference signal for at least one segment of the composite signal, wherein each at least one segment comprises a plurality of single cycles; extract each of the plurality of single cycles from each of the at least one segment from the composite signal and comparing each extracted cycle to the other extracted cycles to identify those signals that have the highest correlation value for the PLI component; determine, concurrently, a signal to noise ratio (SNR) for each of the at least one segment of the composite signal; generate a steady state phase and amplitude of the PLI component based on the determined phase and amplitude of the measured PLI component and a weight-adjusted SNR for each of the at least one segment; and process the composite signal, using at least one of the steady state phase and amplitude of the PLI component or the highest correlation value to remove the PLI component from the composite signal to obtain the PHSI.

Optionally, in determining the SNR for each at least one segment, the PLI component is the signal represented by in-phase power and the PHSI component is the noise represented by out-of-phase power.

Optionally, a steady state PLI is obtained by measuring the PLI at a time when the PHSI is minimal.

Optionally, the processing system is further configured to integrate and filter the PLI component in the composite signal using sequential segments of the composite signal.

Optionally, after integration and filtering, the value of each PLI component is weighted according to its dynamically computed SNR.

Optionally, the processing system is configured to synchronize the reference signal with a sample rate of the measured PLI.

Optionally, the processing system is configured to determine the phase of the measured PLI component using the sine and cosine values of the reference signal.

Optionally, the source of the PLI component is a mains power supply.

Optionally, the PHSI is a bioelectrical signal.

Optionally, the processing system is further configured to: delay the composite signal in order to detect a change in the PLI component; recompute the PLI component; apply the recomputed PLI component retroactively to the composite signal to determine an updated phase and amplitude of the PLI component for each at least one segment, using the PLI component that follows the delayed composite signal; and subtract the updated phase and amplitude of the PLI from the composite signal to obtain the PHSI.

In some other embodiments, the present specification discloses a method for processing a composite signal comprising a PLI component and a PHSI, wherein the method comprises: measuring a PLI from a source of the PLI component, wherein the PLI is an analog signal, and wherein the PLI contributes to the composite signal in the form of the PLI component; generating a reference signal by digitizing the PLI; determining a phase and an amplitude of the PLI component using the generated reference signal, wherein the determining is performed for each segment of a plurality of segments of the composite signal; determining a signal to noise ratio (SNR) for each segment of the plurality of segments; generating a steady state phase and amplitude of the PLI component based on the determined phase and amplitude and a weight-adjusted SNR; processing the composite signal, using the steady state phase and amplitude of the PLI component to removing the PLI component from the composite signal and obtain the PHSI.

Optionally, in determining the SNR for each segment, the PLI component is the signal and the PHSI component is the noise.

Optionally, a steady state PLI is obtained by measuring the PLI at a time when the PHSI is minimal.

Optionally, the PLI component in the composite signal is integrated and filtered using sequential segments of the composite signal.

Optionally, after integration and filtering, the value of each PLI component is weighted according to its dynamically computed SNR.

Optionally, the reference signal is synchronized with a sample rate of the PLI.

Optionally, the reference signal provides sine and cosine values to determine the phase of the PLI component.

Optionally, the PLI is a signal generated by a mains power supply.

Optionally, the PHSI is a bioelectrical signal.

Optionally, the method further includes delaying the composite signal in order to detect a change in the PLI component; recomputing the PLI component; applying the recomputed PLI component retroactively to the composite signal to determine an updated phase and amplitude of the PLI component for each segment, using the PLI component that follows the delayed composite signal; and subtracting the updated phase and amplitude of the PLI from the composite signal to obtain the PHSI.

In some other embodiments, the present specification discloses a method for removing a periodic interference component from a composite signal comprising the periodic interference component and a signal of interest, the method comprising: measuring a reference signal representative of the periodic interference component; generating a digital reference waveform that is phase-locked to the measured reference signal that includes in-phase and quadrature components; acquiring the composite signal containing the periodic interference component and the signal of interest; segmenting the composite signal into time intervals of predetermined duration; determining, for each segment, a phase and an amplitude of the periodic interference component by correlating the composite signal with the in-phase and quadrature components of the reference waveform; computing a quality metric for each segment based on a relationship between the interference component and the signal of interest; calculating a weighted estimate of a steady-state phase and amplitude of the periodic interference component using the quality metric; monitoring the composite signal over successive intervals to detect a change in amplitude, phase, or quality metric of the interference component; upon detection of a change, updating the reference waveform and re-computing the steady-state phase and amplitude of the interference component using subsequent segments exhibiting higher signal quality; and synthesizing an interference component using the steady-state or updated phase and amplitude, and subtracting the synthesized interference component from the composite signal to obtain the signal of interest substantially free of interference.

Optionally, the predetermined duration is approximately 100 milliseconds, corresponding to five cycles at 50 Hz or six cycles at 60 Hz.

Optionally, the quality metric is signal-to-noise ratio.

The aforementioned and other embodiments of the present specification shall be described in greater depth in the drawings and detailed description provided below.

### BRIEF DESCRIPTION OF THE DRAWINGS

The accompanying drawings illustrate various embodiments of systems, methods, and embodiments of various other aspects of the disclosure. Any person with ordinary skills in the art will appreciate that the illustrated element boundaries (e.g. boxes, groups of boxes, or other shapes) in the figures represent one example of the boundaries. It may be that in some examples one element may be designed as multiple elements or that multiple elements may be designed as one element. In some examples, an element shown as an internal component of one element may be implemented as an external component in another and vice versa. Furthermore, elements may not be drawn to scale. Non-limiting and non-exhaustive descriptions are described with reference to the following drawings. The components in the figures are not necessarily to scale, emphasis instead being placed upon illustrating principles.

FIG. 1 illustrates an exemplary waveform displaying at lease a mains signal, a power line interference (PLI) component generated by the mains signal, a physiologic signal of interest (PHSI), and a composite signal (PHSI plus PLI), in accordance with embodiments of the present specification;

FIG. 2 is an exemplary process flow chart, which is implemented by some embodiments of the present specification;

FIG. 3A illustrates an exemplary FFT graph of a clean composite signal consisting of mains noise without bioelectric signal as shown in FIG. 1;

FIG. 3B illustrates an exemplary FFT graph of a dirty composite signal where mains and bioelectric signals are both present as shown in FIG. 1; and

FIG. 4 provides another illustration of the process of FIG. 2 with reference to and in the context of system components, in accordance with some embodiments.

### DETAILED DESCRIPTION

The present specification is directed toward removal of power line interference (PLI) from at least one physiologic signal of interest (PHSI) where the PLI and the PHSI occur simultaneously. The PLI can be generated within an environment where one or more devices are operating to record the PHSI. The PLI may also be referred to as noise or mains interference signal. While there are several different types of noises that are known to disrupt signals of interest, embodiments of the present specification are concerned primarily with interference from power line interference (PLI). Therefore, for the purposes of the present specification, PLI refers to the signals caused by the electric and magnetic field in the vicinity of a device that is recording the PHSI.

It should be appreciated that the disclosed method of the present specification is particularly advantageous in applications where conventional techniques for managing power line interference (PLI) are not optimal.

The physiologic signals of interest (PHSI) refer to bioelectrical signals that originate from electrical phenomena that take place on cell membranes of living organisms. The sum of action potentials at different anatomical sites are recorded as bioelectrical signals by devices designed to be sensitive to such signals, for clinical and medical monitoring purposes. These devices are used with stimulation of electrical current at the anatomical site, which is then monitored for electrical activity. Different types of bioelectrical signals can include, and are not limited to Electroencephalogram (EEG), Electrocorticogram (ECOG), Evoked Potentials (EP), Electrooculogram (EOG), Electroretinogram (ERG), Electromyogram (EMG), Electrocardiogram (ECG), Electrogastrogram (EGG), Vectorcardiogram (VCG), Electrohysterogram (EHG), Electroneurogram (ENG) and Fetal ECG (fECG).

The present specification is directed towards multiple embodiments. The following disclosure is provided in order to enable a person having ordinary skill in the art to practice the invention. Language used in this specification should not be interpreted as a general disavowal of any one specific embodiment or used to limit the claims beyond the meaning of the terms used therein. The general principles defined herein may be applied to other embodiments and applications without departing from the spirit and scope of the invention. Also, the terminology and phraseology used is for the purpose of describing exemplary embodiments and should not be considered limiting. Thus, the present invention is to be accorded the widest scope encompassing numerous alternatives, modifications and equivalents consistent with the principles and features disclosed. For purpose of clarity, details relating to technical material that is known in the technical fields related to the invention have not been described in detail so as not to unnecessarily obscure the present invention.

In the description and claims of the application, each of the words “comprise”, “include”, “have”, “contain”, and forms thereof, are not necessarily limited to members in a list with which the words may be associated. Thus, they are intended to be equivalent in meaning and be open-ended in that an item or items following any one of these words is not meant to be an exhaustive listing of such item or items, or meant to be limited to only the listed item or items. It should be noted herein that any feature or component described in association with a specific embodiment may be used and implemented with any other embodiment unless clearly indicated otherwise.

It should also be noted that as used herein and in the appended claims, the singular forms “a,” “an,” and “the” include plural references unless the context dictates otherwise. Although any systems and methods similar or equivalent to those described herein can be used in the practice or testing of embodiments of the present disclosure, the preferred systems and methods are now described.

In various embodiments, a computing device includes an input/output controller, at least one communications interface and system memory. The system memory includes at least one random access memory (RAM) and at least one read-only memory (ROM). These elements are in communication with a central processing unit (CPU) to enable operation of the computing device. In various embodiments, the computing device may be a conventional standalone computer or alternatively, the functions of the computing device may be distributed across multiple computer systems and architectures.

In some embodiments, execution of a plurality of sequences of programmatic instructions or code enable or cause the CPU of the computing device to perform various functions and processes. In alternate embodiments, hard-wired circuitry may be used in place of, or in combination with, software instructions for implementation of the processes of systems and methods described in this application. Thus, the systems and methods described are not limited to any specific combination of hardware and software.

The term “module”, “application”, “component” or “engine” used in this disclosure may refer to computer logic utilized to provide a desired functionality, service or operation by programming or controlling a general purpose processor. Stated differently, in some embodiments, a module, application, or engine implements and/or is configured to implement a plurality of instructions or programmatic code to cause a general purpose processor to perform one or more functions. In various embodiments, a module, application or engine can be implemented in hardware, firmware, software or any combination thereof. The module, application or engine may be interchangeably used with unit, logic, logical block, component, or circuit, for example. The module, application or engine may be the minimum unit, or part thereof, which performs one or more particular functions. It should be noted herein that each hardware component is configured to perform or implement the plurality of instructions or programmatic code to which it is associated, but not limited to such functions.

In embodiments of the present specification, a “single cycle” refers to one complete period of the mains-frequency interference waveform as it appears within the composite signal that contains both the PLI and the PHSI. If the mains interference is a 50 or 60 Hz sinusoid, a single cycle is the continuous portion of the composite signal corresponding to one full repeat of that sinusoid (from a defined phase point back to the same phase point again). The system uses the measured mains frequency and a phase-locked digital reference to locate these boundaries in time and then extracts one-cycle windows from the composite signal. Each extracted single cycle thus contains the PLI component over exactly one interference period, plus whatever PHSI happens to be present during that time. These single-cycle snippets are then compared (correlated) with each other; cycles whose waveforms match most closely (highest correlation) are treated as cleanest examples of the mains interference, while poorly correlated cycles are treated as being more contaminated by PHSI and are excluded from representing the PLI when synthesizing the interference waveform to subtract.

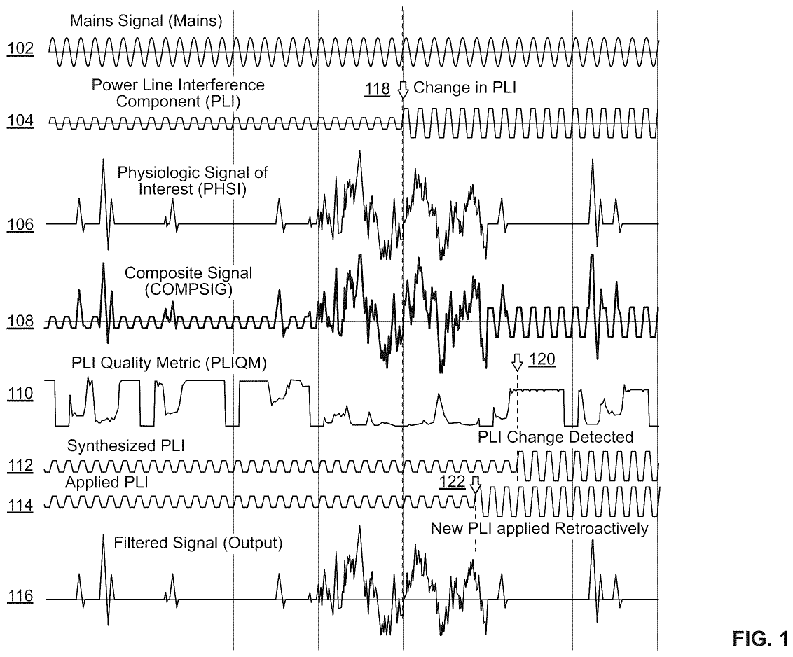

FIG. 1 illustrates an exemplary waveform of a mains signal 102, a power line interference (PLI) 104 generated by the mains signal 102, a physiologic signal of interest 106 (PHSI), a composite signal 108 (COMPSIG) consisting of the PHSI and PLI, a graphic illustration of a quality metric 110 (PLIQM) used in estimating the underlying PLI, and a synthesized PLI 112. Also shown is how the synthesized PLI 112 is applied retroactively 114, and the resulting filtered signal 116 (Output) in accordance with the present specification.

In embodiments, the present specification describes advantageously recording the mains signal as a reference signal and separately recording the composite signal (which is the mains derived PLI plus PHSI), and then using the reference signal as a steady measure of the noise or interference to filter the target signal (which is the physiologic signal of interest, or PHSI).

The optimal synthesized PHSI signal to remove from the ongoing signal depends on both the PHSI signal and the level of physiologic activity over time. In embodiments, the optimal solution is selected dynamically and includes, but is not limited to the currently extracted PHSI, the PHSI from previous segments, the PHSI from future segments, as well as no PHSI when the PHSI is small, an alternate filter (e.g. conventional notch filter), or a combination of algorithms and AI.

The mains signal and the PLI generated by the mains signal are frequency locked by definition and have a constant phase relation to each other. This allows using the detected main signal as a more robust method to analyze the PLI contained in the combined signal. Thus, according to embodiments of the present specification, the mains signal is physically detected. This can be accomplished with a zero-crossing detector (which, in some embodiments is an electronic circuit, for example, that detects the exact instant a periodic waveform crosses zero volts; in other words, when it transitions from positive to negative or negative to positive. The circuit is configured to convert these zero-crossing events into timing information (digital pulses), which can be used to measure frequency, period, or phase of the waveform) or with a phase-locked loop. The process of extraction of the phase and frequency of the PLI directly from the supply contributes a significant advantage over the known methods of removing PLI where the interference signal is extracted from the composite signal.

The PLI superimposes upon the co-occurring PHSI, resulting in contamination of the PHSI, which is the signal recorded by an amplifier (referred to herein as the composite signal 108) of a neuro-physiology monitoring system. The fundamental component in the PLI is at the mains frequency (typically 50 Hz or 60 Hz) and may contain sinusoidal harmonic frequency components at multiples of 50 Hz or 60 Hz.

In embodiments, the PLI component in the composite signal is analyzed in fixed-duration segments to derive its phase and amplitude relationship to a phase-locked reference signal, where the segment duration Tis chosen so that each segment contains an integer number of interference periods and thus multiple back-to-back single cycles of the mains frequency PLI plus any concurrent PHSI (for example, 100 ms segments containing five 50 Hz cycles or six 60 Hz cycles). A “single cycle” in this context is the portion of the composite signal corresponding to one complete period of the PLI waveform, and because the PLI contribution is tightly phase-locked and fully correlated with the reference while the PHSI contribution is uncorrelated. Single-cycle correlation values are high when the composite signal contains only PLI (successive cycles look nearly identical) and lower when PHSI is present (cycles vary in shape); accordingly, the system measures the PLI cycle time with high-resolution timing (for example, via a zero-crossing detector with microsecond resolution), applies frequency-analysis techniques within each segment (such as frequency response, time-frequency analysis, or oscillators) to estimate PLI phase and amplitude by cross-correlating the composite signal with in-phase and quadrature versions of the reference, and then treats highly correlated single cycles as primarily mains components while treating poorly correlated cycles as mains plus PHSI that are excluded from representing the PLI.

In embodiments, the present specification is directed towards systems and methods for removing or compensating for the transient artifact in a composite signal by using an actual PLI (detected from the mains signal) rather than a surrogate or estimated PLI that is extracted from the composite signal. The PLI is also measured periodically with high precision (accurate within 1% and repeatable within 0.1%) to generate a reference signal that has a stable phase and frequency. The PLI component in the composite signal is analyzed in segments to derive the phase and amplitude relationship with the reference signal. In embodiments, machine learning methods of the present specification are used to identify periods of low activity when the PHSI is either low or absent. The analysis results in a quality metric (PLIQM) which dynamically reflects the quality of the PLI component of the combined signal. During such periods of low PHSI activity, the measured composite signal power sets a baseline for noise or interference from the ongoing PLI. Further, during periods of activity when PHSI are present, the power of the composite signal increases due to contributions by both the PLI and the PHSI. The power contributed by the PLI has a precise and constant phase and 100% of the signal is correlated to the noise component of the combined signal. Conversely, the power contribution of the PHSI is uncorrelated to the reference signal.

In embodiments, a segmental analysis of an ongoing composite signal is performed wherein trained algorithms are used to detect the interference from the PLI and remove it from the composite signal to obtain a filtered output 116 which contains just the physiologic signal of interest (PHSI), in real time. In an embodiment of the present specification, the waveshapes of the PHSI are preserved while eliminating the interference from power line interference (PLI) and the resulting harmonics, from the composite signals. This is especially significant since the PHSI may come and go in bursts and may include frequency content at the frequency of the PLI itself or at the harmonics level of the PLI.

It should be noted herein that PLI encompasses several attributes such as: amplitude, phase, and content of harmonics. Depending on the parameters of the PLI, some methods of removal are more effective than others. In some other embodiments of the present specification, machine learning algorithms are used to train and configure a computing device to determine when and how to apply different filter techniques to eliminate the PLI, and when and how to update the filter parameters when the PLI changes, especially abruptly. Therefore, some embodiments of the present specification adopt a strategy based on the requirements, whereby the strategy may or may not include the use of a filter.

In embodiments, the present specification additionally addresses situations where the higher frequency harmonics described above are absent, and therefore filters to remove them are not required. PHSI are cleaner when the filters to remove higher frequency harmonics are not applied (when the higher frequency harmonics are absent), wherein the filters would otherwise generate an interference in the form of a buzz.

The elimination methodology for the first and subsequent harmonics includes no removal, removal of synthesized sinusoidal components, removal of optimal extracted PLI components, or a notch filter at the frequency of the harmonic. The source of the signal can be prior to an event or noise transition, or after the event or noise transition. The methodology for artifact reduction for each segment is determined by an AI algorithm that maximally reduces visual artifact, wherein inputs to the algorithm are the mains phase and frequency, the binned FFT (Fast Fourier Transform) values that determine the mix of a first signal level (PLI synchronous with the mains signal) and a second signal level (PHSI which is not synchronous with the mains signal), the values of the segments preceding and following the segment being filtered, the relative ratios of signal and noise, and the elapsed interval since the last optimal PLI signal was synthesized.

The characteristics of power line interference (PLI) and physiological signals of interest (PHSI) described below are considered in embodiments of the present specification to adapt a filter strategy. It should be noted that the phase, amplitude and waveshape of the PLI first signal is synchronous with and correlated to the mains signal, and repeats at the mains frequency. The PHSI second signal is asynchronous to the mains signal and varies independently from the mains signal.

A filter strategy should consider whether the frequency of the PLI is stable within fractions of a Hz over long intervals of time. In some cases, published data from third world power utilities claim an observed approximate variation of 0.05 Hz in the frequency over minutes to hours.

It should be determined whether the noise or interference associated with a composite signal, as picked up by a neurophysiology equipment, has the same frequency as that of the PLI. The phase of the PLI and the noise component of the PHSI may differ in the composite signal but have a constant relation.

It should further be determined whether the stability and synchronicity of the PLI with the component of the PLI in the composite signal, allow for detection and characterization of the PLI component.

A filter strategy should also consider whether the amplitude of the PLI picked up by the neurophysiology equipment (component of the PLI in the composite signal) remains constant until an electrode is moved, a new device is activated, or some other non-transient change occurs. After any of the changes (an electrode is moved, a new device is activated, or some other non-transient change) occur, it should be determined whether the PLI component in the composite signal is again stable at a new level.

It should also be determined whether changes in the component of the composite signal contributed by the PLI can be detected and compared with subsequent segmental PLI components and whether the results can be applied retroactively, to eliminate settling time and ringing.

EMG, among other neurophysiological signals, has attributes that support the utility of an AI-driven reduction of the PLI contribution in the composite signal especially due to a constant coupling with the source of the PLI and that the interference contributed by abrupt changes in the PLI is preceded and followed by long periods of constancy. In addition, periods of low neurophysiological signal activity exist during which the contribution of the PLI can be determined without artifact from the PHSI. The PLI contribution changes with changes in needle position, but very consistently repeats at the mains frequency, at either 16.6 msec or 20 msec. As a result, the PLI in the combined signal can be determined accurately when the physiological signal of interest is small or absent.

Therefore, in the exemplary case of EMG, a needle exam is characterized by periodic abrupt needle movement which adds movement artifact that may also change the detected PLI. The abrupt needle movement is followed by one or two seconds of non-movement while a user listens and looks for physiological effects of the movement. The resulting signal (or “composite signal” which is the composite of the PLI and the PHSI) seen during this time is referred to as ‘insertional activity’. However, the abrupt movements generate large transient artifacts within the composite signal, and because the resulting impedance may be different in the new location due to the transient artifact, the level of interference in the composite signal contributed by the PLI, may change significantly.

In conventional filtering methods that use a notch filter, the transient artifacts have a high energy content (on the order of 10-100 m Volts) that will perturb the notch resonator. The notch resonator, which is tuned to reject a specific frequency, is disturbed by the transient artifact which may result in a short period of time when the notch filter rejects a portion of the PHSI and allows a portion of the interference signal. The notch resonator requires time to recover from the disturbance caused by the transient artifact. Moreover, the subsequent interference level contributed by the PLI after the transient artifact may be different than the previous level, which also adds to the time required by the composite signal to settle at a consistent level. While settling, the notch filter artifact is added to the composite signal instead of being subtracted. The addition occurs during the several hundred milliseconds that are the most important for observing the insertional activity.

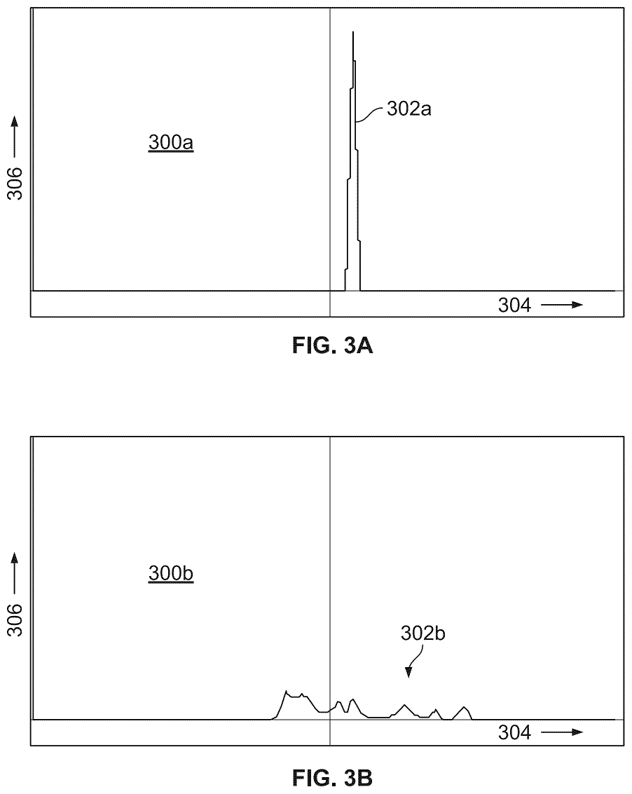

In some embodiments, a quality metric or signal to noise ratio (PLIQM) is obtained, wherein the signal is the PLI (which contributes the noise or interference) and the noise is the PHSI. The PLI is considered constant over periods of many seconds as long as the recording input does not change, but the PHSI increases and decreases as the subject moves, as muscles contract, when the heart beats or a stimulus is applied. The background PLI or the ‘steady state’ PLI is obtained by measuring the PLI at a time when the PHSI is minimal. Minimal PHSI is determined with at least one of two metrics: 1) the ratio of in-phase to out-of-phase signal (higher is better) and 2) the number of highly correlated cycles in a segment (higher is better). The FFT 302a, shown in FIG. 3A, has a single peak at the mains frequency when this occurs. The noise floor is used to detect steady state changes. Therefore, the present specification uses an inverse form of the SNR compared to traditional methods. The PLIQM is normalized between 0 and 1. In the present specification, the PLIQM is obtained over sequential segments of the composite signal by comparing the now known lower limit to the actual value. The PLI is phase and frequency stable, and the PHSI, which may contain frequencies near the PLI, has a variable phase relation to the PLI, allowing separation of the two signals from the composite signal. The correlation values of single cycle signals is high when only PLI is present as all cycles are nearly the same, but will be lower if PHSI is present as the single cycle signals will vary.

The PLI component in the composite signal is integrated and filtered. The process of collection of the PLI component in the composite signal is performed from sequential segments of the composite signal. The value of each PLI component, after integration and filtering, is weighted according to its dynamically computed PLIQM value. Therefore, ‘mostly pure’ PLI components from the composite signal are heavily weighted and the PLI contaminated with bioelectric activity is weighted less heavily. Additionally, composite signals that contain activity in the PHSI, which have a low SNR, do not affect the calculation of the PLI component.

Alternatively, the composite signal can be segmented into single cycle intervals using the mains frequency detector, and the ongoing cycles correlated with each other around the mains frequency. Highly correlated cycles are presumed to be PLI, which are primarily mains components and poorly correlated cycles are presumed to be mains plus PHSI and are removed from consideration as PLI representatives.

A computation network that is trained using machine learning evaluates both the phase and phase quality of the PLI component in the composite signal. The phase and phase quality evaluations are performed over multiple contiguous segments of the PLI component. The evaluation is used to specify the most likely phase and amplitude of the PLI in each segment. The phase and amplitude information of the PLI component are used by a synthesizer to create a replica of the PLI. The synthesized signal is subtracted from an incoming composite signal to create a PHSI that is free from the PLI. As mentioned above, in some instances, the amount of PLI may change with needle movement and the synthesized PLI component may no longer be correct. The derived PHSI is delayed allowing for computation of both preceding and following estimates of the PLI, and the best estimate is retroactively applied to the signal before display (shown as 122 in FIG. 1). The best estimate for each segment is an average of the highly correlated cycles.

In using the above methods for processing the composite signal, when the PHSI is active, the contribution of the PLI frequency component to the composite signal will be the same. The phase relation of the PLI within the composite signal may change over a duration of time, but the phase relation is more similar than different. The harmonics contributed by the PLI component in the composite signal remain at the same frequency. The amplitude of the noise or interference contributed by the PLI component is at a new steady state value. Embodiments of the analysis of above-described methods can also be achieved for harmonics of the PLI to remove harmonically related noise.

Arithmetically the relationships among the traces shown in FIG. 1 are:

$$PLI = Function ( Mains )$$

$$Composite Signal = PLI + PHSI$$

$$PLIQM ∼ PLI / PHSI$$

$$Synthesized PLI = Function ( estimated PLI phase and amplitude at harmonic intervals )$$

$$Output ( Filtered Signal ) = Composite Signal - Synthesized PLI$$

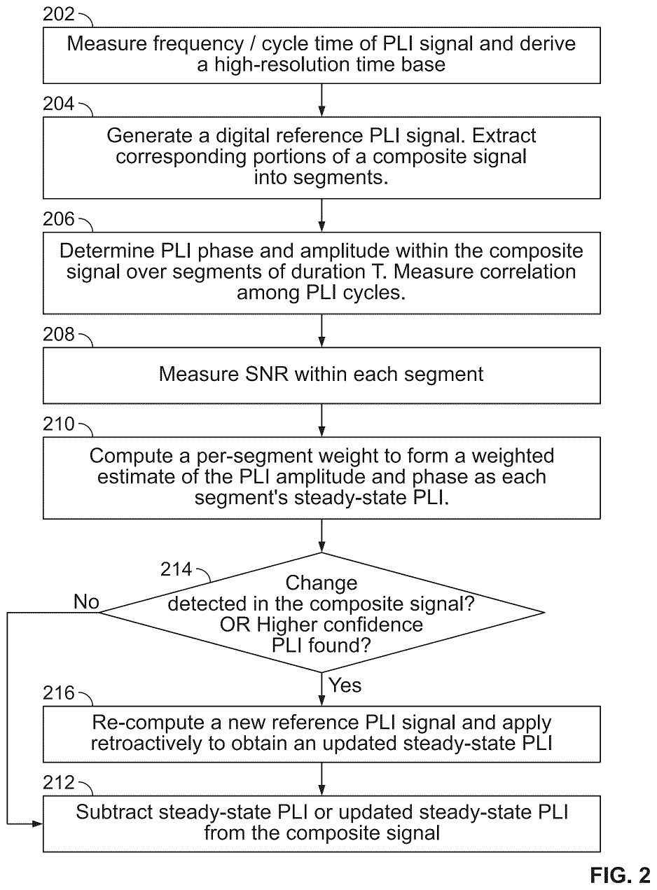

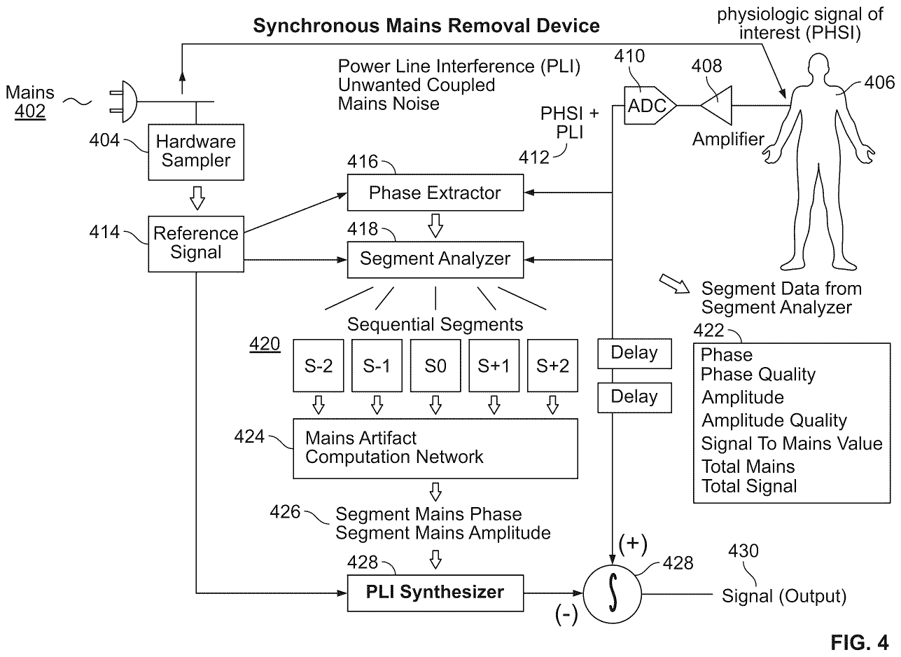

FIG. 2 shows an exemplary process flow for some embodiments of the method of the present specification. FIG. 4 provides another illustration of the process of FIG. 2 with reference to system components, in accordance with some embodiments. Referring simultaneously to FIGS. 2 and 4, at step 202, a frequency of the PLI signal is measured. The PLI is, in embodiments, an analog signal from a mains power source 402. It should be noted that signals from the mains power source comprise a voltage waveform which is used by electrical and electronic devices, such as bioelectrical signal measurement devices used to measure bioelectrical signals on a person 406, to connect to a power grid and derive electrical energy for operation of the device. For purposes of the present specification, it is assumed that the frequency of the PLI that is measured is stable and drift is small over long intervals. In most cases, a ‘stable’ signal refers to a signal with a drift in its frequency of less than 0.1 Hz over a 1-minute interval.

Simultaneously, at least one measured bioelectrical signal (PHSI) from a person 406 is amplified using an amplifier 408. The amplified signal is converted to a digital signal using an analog-to-digital converter 410. A resulting signal 412, herein referred to as a composite signal, is a combination of the measured bioelectrical signal (PHSI) by a neuro-physiology device and the interference due to the presence of the mains signal (PLI) from source 402.

In some embodiments, a cycle time (time taken to complete exactly one cycle) of the PLI signal is measured with a zero-crossing detector. In alternate embodiments, any other precise frequency measurement hardware is used to measure the cycle time of the PLI signal. The measured cycle time uses nominal 1 microsecond resolution each for a high-resolution time base.

At step 204, a digital reference PLI signal 414 is generated using the measured analog PLI signal. The reference PLI signal 414 is generated by periodically measuring the PLI signal from the mains using a sampler 404. The measurement is performed with high precision. The resulting reference signal 414 is a high-resolution digital signal of the same frequency as the measured PLI and is generated at the same sample rate (i.e. ticks or increments on the order of 1 microsecond) at the measured signal.

In some embodiments, the generation of the digital reference PLI signal 414 further includes extracting corresponding portions of the composite signal 412 into a plurality of fixed-duration segments based on the microsecond-resolution increment time base (i.e. sample rate). Each segment is temporally aligned with the reference PLI signal so that operations, such as phase correlation, in-phase and quadrature extraction, and segment-level amplitude estimation, are performed on time-synchronized data.

The sample rate used to digitize the mains-derived PLI signal and the sample rate used to acquire the composite signal 412 are typically not integer relations and each may exhibit frequency drift. As a result, the number of acquisition samples corresponding to a single cycle of the mains waveform is not an integer value; for example, at a nominal 60 Hz mains frequency and a nominal acquisition sampling rate, a single PLI cycle may span approximately 153 samples, with fractional-sample offsets varying over time due to drift. To address this, the system aligns the digital reference PLI signal 414 to the acquisition time base in microsecond-resolution increments by adjusting the placement of the reference samples so that each data point of the digital PLI corresponds as closely as possible to the true temporal position of the mains waveform. In some embodiments, the fractional alignment is performed using a running average or continuously updated estimate of the mains frequency, allowing gradual frequency drift to be tracked without introducing discontinuities in phase. Importantly, the alignment process ensures that no data point is ever skipped or duplicated, preserving a continuous, phase-accurate digital reconstruction of the PLI signal. Thus, while sampler 404 provides the high-precision measurements used to construct the reference signal, the effective sample rate of the digital reference PLI signal is governed by the acquisition time base and the incremental microsecond tick resolution, not by the nominal sampling frequency of sampler 404 alone.

The digital reference signal 414 (PLI) generates the sine (in phase) and cosine (out of phase) values for “other signals” whose time is also measured in ticks, where these values are used subsequently as described in the present specification. Stated differently, the digital reference PLI signal 414 is used to generate a pair of orthogonal reference waveforms—that is, a sine waveform representing the in-phase component (0° phase) and a cosine waveform representing the out-of-phase or quadrature component (90° phase shift). Thus, the digital reference PLI signal 414 functions as a precise timing standard derived from the mains waveform, producing a microsecond-resolution representation of the PLI phase for each mains cycle. In the sample-rate time scale, successive mains cycles are almost, but not exactly, contiguous with the acquisition samples of the composite signal 412 due to fractional-sample relationships and minor clock drift. By allowing the reference PLI waveform to be shifted by as little as a single acquisition-sample interval to maintain precise temporal alignment, the cancellation of the PLI artifact is substantially improved, often by many decibels. This fine-grained alignment is particularly significant for higher-order harmonics of the mains frequency, for which even small timing errors can materially degrade rejection performance. The term “other signals” refers to signals whose samples share this same microsecond-based time base, including, most importantly, the composite signal 412 and any intermediate signals that may be derived from it during, for example, segmentation. By synchronizing these signals to the timing of the reference PLI signal, the system ensures accurate alignment for operations related to segment analysis at 418 and PLI synthesis at 428.

At step 206, the phase and amplitude of the PLI component in the composite signal 412 is determined. The phase is extracted at stage 416. The amplitude of the PLI component in composite signal 412 is also extracted. If the neuro-physiology device that is being used to record the PHSI is battery operated, the PLI component in the composite signal 412 generated by the device can be detected with a high impedance sensing circuit and conditioned to provide a high-resolution representation of the PLI component. The composite signal 412 is a composite of the noise or interference from the PLI (herein termed as ‘PLI component’) and the PHSI (or the bioelectrical signal component).

In some embodiments, step 206 further includes measuring correlation values among individual PLI cycles within each segment to assess the internal consistency of the PLI waveform. The correlation value measurement is performed after removing DC and low frequency components. Pure PLI cycles will have a correlation value of 1, composite signals will have variable and mostly random correlation values which may include values close to 1. The highly correlated signals are then binned by amplitude to exclude randomly included signals that do not represent the PLI. In addition, signal-to-noise ratio (SNR) measurements are computed for each segment and used in conjunction with the correlation values to reject contaminated or unreliable PLI candidates. A confidence metric is computed from the combined correlation and SNR assessments to determine whether successive cycles exhibit stable, repeatable characteristics. High cycle-to-cycle correlation indicates a clean PLI component within the segment, whereas poor correlation indicates distortion, drift, or contamination from the PHSI. Once a high-confidence PLI estimate is identified, in embodiments, the corresponding phase and amplitude is retained and reused to remove PLI artifacts from subsequent segments that contain high PHSI content, until a new high-confidence PLI estimate is detected and adopted. Poor correlation or low SNR indicates distortion, drift, or contamination from the PHSI and results in reduced confidence or rejection of the candidate PLI cycles.

In embodiments, the highest correlation (confidence) value is determined using individual single PLI cycles within the segment of the composite signal. The correlation values are used to determine internal consistency of the PLI waveform. The weight is used to determine, for a segment's SNR and power, how strongly a segment should influence the steady-state PLI estimate.

At 416 and 418, one of the known frequency analysis techniques, including and not limited to—frequency response analysis, time-frequency analysis or oscillators—is used to determine the phase and the amplitude of the PLI component in exact time intervals or segments of duration ‘T’ of the composite signal 412. In some embodiments, T is 100 milliseconds (msec). The value of T is selected so that each segment contains a whole number of cycles of the signal, and preferably multiple back-to-back single cycles of the periodic interference (PLI) plus the physiological signal (PHSI). If T is 100 msec, each segment will contain 5 cycles (at 50 Hz of the PLI) or 6 cycles (at 60 Hz of the PLI). At stage 416, the phase of the PLI component within composite signal 412 is detected relative to the phase of reference signal 414. In some embodiments, this is achieved by cross correlating the PLI component with a 0-degree phase shift and a 90-degree phase shift from the reference signal. Known mathematical methods are used to compute the phase of the PLI component in the composite signal.

In an exemplary phase analysis method, an in-phase waveform of the PLI (with 0-degree phase shift) is determined as follows:
A. First, an in-phase waveform is computed as a composite signal x in-phase reference signal. This is primarily the contribution of the PLI to the composite signal plus any in-phase component of the PHSI that is present.
B. Second, an out-of-phase waveform is computed as a composite signal x out-of-phase reference signal. This result is indicative exclusively of the PHSI.
C. Third, a power waveform is computed as a square of the combined voltage.
   The phase analysis generates a confidence level: high confidence is when the in-phase waveform is greater than the out-of-phase waveform, and the power waveform is greater than zero. The operational phase of the reference signal is updated if the confidence is high.

In an exemplary cycle analysis, a moving rectangular window having a duration of one cycle (approximately 16.6 msec for 60 Hz or 20 msec for 50 Hz of the PLI) within the PLI component, generates a moving average for the following:
D. Total power using the power waveform (computed at C as the square of voltage).
E. In-phase cycle voltage using in-phase waveform computed at A. (to calculate C and D).
F. Out of phase cycle voltage using out-of-phase waveform computed at B.
G. In phase cycle power is determined as a square of in-phase cycle voltage (computed at E). This computation is representative of the PLI component and is used as the Signal(S) to subsequently determine the signal-to-noise ratio (SNR).
H. Out of phase cycle power is determined as a square of out of phase cycle voltage (computed at F). This computation is representative of the PHSI component in the composite signal and is used as the Noise (N) to subsequently determine the signal-to-noise ratio (SNR).

The phase and amplitude analysis is performed in each segment at 418. The output or outcome of the segment analysis is a sequence of segments 420 of the PLI component. Each segment in the sequence of segments 420 contains data from the analysis of that segment, and includes information 422 pertaining the PLI component that may be related to: phase, phase quality, amplitude, amplitude quality, ratio of PHSI to PLI component, total PLI value, and the total value of the PHSI component. The sequence of segments 420 comprising the PLI component(S) and the PHSI component (N) in the composite signal are input into a computational network 424.

At step 208, the SNR value is measured for each segment of time T. In the present specification, the PLI component is considered to be the signal (S in SNR), and the PHSI component is considered to be the noise (N in SNR) as described above.

At step 210, a weight is calculated for each segment as follows: SNR/total cycle power (where the total power is calculated at D). The SNR of each segment is adjusted using the weight calculated for that segment. The weight is a measure of the extent of similarity of the PLI component in that segment to the reference signal. SNR for each segment is therefore weight-adjusted to arrive at a steady state PLI component for that segment (shown at 426 in FIG. 4).

Voltages are binned using the in-phase cycle voltage (where the in-phase cycle voltage is calculated at E.) The PLI component has a unique value at each data point. These values are binned to find the most common voltage and distribution. A clean composite signal comprising mostly the PLI component, and which has a minimal or an absence of the PHSI, will fall into a single bin or a few closely-timed bins.

Thus, for each segment of the composite signal (for example, each 100 msec segment), the system is configured to compute SNR where the signal(S) is the in-phase cycle power (power of the PLI component) and the noise (N) is the out-of-phase cycle power (power of the PHSI component). The composite signal is correlated with a reference PLI signal (sine and cosine) to obtain the in-phase and out-of-phase waveforms. For each single cycle of the PLI within the segment, the in-phase cycle voltage and the out-of-phase cycle voltages are computed from the respective waveform. The signal(S) is calculated as the square of the in-phase cycle voltage, which represents the in-phase cycle power. The noise (N) is calculated as the square of the out-of-phase cycle voltage, which represents the out-of-phase cycle power. The SNR for that segment is the ratio of S/N (i.e. the ratio of PLI power to PHSI power in that segment). The system is configured to then compute a weight for each segment as follows: SNR/total cycle power, where SNR is the segment's SNR and total cycle power is the total power of the composite signal in that cycle (the square of the composite voltage). The weight is the measure of the extent of similarity of the PLI component in that segment to the reference signal. A segment is considered similar if its in-phase waveform closely follows the reference sine wave, while the out-of-phase component is small. In other words, the weight is a number that reflects how strong the PLI is relative to PHSI (via SNR) and how much of the total signal energy is “clean” PLI versus contaminated PHSI (via total power). The weight-adjusted SNR is obtained by adjusting the raw SNR using this weight.

FIG. 3A is an exemplary graph 300a of a clean signal. The clean signal 302a has a sharp peak which is indicative of a high confidence in the value of the signal to determine the PLI value. FIG. 3B is an exemplary graph 300b of a dirty composite signal. The dirty signal 302b has many more bins across multiple voltage levels and none of the bins appear to be prominent. Signal 302b is indicative of presence of PHSI (noise) in the form of insertional activity, along with the PLI component. Signal 302b is indicative of a low confidence in the signal to use for the determination of the PLI value. Both graphs 300a and 300b illustrate the bin frequency value along their x-axis 304 and bin count along y-axis 306. The most common in-phase voltage is indicated by the largest bin along x-axis 304, and the count (along y-axis 306) of the largest bin is determined to provide the highest confidence.

In some embodiments, the in-phase cycle voltage (calculated in E) is normalized using the best voltage. The best voltage is the voltage corresponding to the bin with the highest confidence. The normalization process limits the voltage slew rate. Thus, based on the computed SNR, the PLI component's amplitude and phase are updated with sample weights between 0 and 1, generating steady state mains amplitude and phase values 426.

The resulting segmental signals with steady state mains amplitude and phase values for the PLI component (steady state PLI 426) are synthesized (see step 428) with the composite signal 412. Specifically, at step 212, the steady state PLI is subtracted from the composite signal using a notch filter. The filter output is identified as the PHSI 430 that is absent of any noise, interference, or influence of the PLI.

In embodiments, the systems of the present specification are configured to monitor for an occurrence of a change in the composite signal 412. A determination of the occurrence of a change is made in parallel to the previous steps from 202 to 210. The PLI component comprises a sinusoidal or a distorted sinusoidal signal at a specified frequency (50 Hz or 60 Hz). In the distorted sinusoidal signal, while the frequency is stable, the amplitude may vary abruptly, resulting in abrupt changes in the composite signal 412. Additionally, other factors may result in an abrupt change in the composite signal 412, which may include movement of an electrode, activation of a new device in proximity of the original device being used to identify the PHSI, or some other non-transient change.

In some embodiments, at step 214, each predefined interval of the composite signal 412 is delayed, which supports using future PLI values on the current signal, wherein the current signal refers to the presently buffered segment of the composite signal 412 for which PLI removal is being performed, and whose output is delayed to permit the application of PLI phase and amplitude parameters derived from one or more subsequently acquired segments. Each predefined interval of the composite signal is also monitored in step 214 to support identification of an abrupt change to the PLI component of the composite signal 412. In some embodiments, the predefined interval ranges from 100 msec to 300 msec. In some embodiments, a predefined interval of 300 msec is used. In some embodiments, each interval is analyzed for abrupt changes in amplitude, phase, and/or signal-to-noise ratio (SNR) relative to the preceding interval. A detected deviation that exceeds a predetermined threshold indicates a transient event or abrupt change in the composite signal 412, suggesting that the phase and/or amplitude of the PLI component may have altered.

In some embodiments, step 214 further includes determining whether a newly acquired segment exhibits a higher-confidence PLI component than previously observed, based on metrics such as, but not limited to, SNR and the fraction of highly correlated cycles.

If no change in the composite signal is identified then the process flow moves to step 212. However, if a change in the composite signal is identified or when a segment with higher confidence is identified then the process flow moves to step 216 prior to moving to step 212. Confidence is based on numerical analysis of each segment, the difference between a current PLI value of high confidence and a previous steady-state PLI value, and the interval (number of segments) since a new steady-state PLI value was selected. The difference (or similarity) is the correlation between the current and previous segmental PLI's. The delay factor is a non-linear decrease in segmental confidence applied to the previous PLI.

At step 216, when a change in the level of the composite signal 412 is detected, the PLI component in the composite signal 412 that follows the signal of interest is used to immediately recompute a new reference PLI signal 414, which is applied retroactively. If the composite signal has a low SNR, then the next good SNR segment is used to adjust previous segments, in embodiments. The new reference signal 414 is used to perform the computations described in steps 206 to 210. Stated differently, a new reference PLI signal 414 is immediately recomputed using one or more subsequent segments of the composite signal 412 that exhibit a high SNR or minimal physiologic signal content. The newly computed reference PLI signal 414 is applied retroactively to reprocess earlier affected segments by repeating the computations of steps 206 through 210 to obtain an updated steady-state PLI 426.

When a segment with higher confidence is identified, the steady-state PLI is updated using the phase and amplitude values from that segment, even in the absence of an abrupt change in the composite signal. In some embodiments, when the PHSI content within the composite signal is low, the steady-state PLI may be updated on a per-segment basis.

Consequently, the steady state PLI 426, whether original or updated after identifying an abrupt change in the composite signal 412, is used to subtract from the composite signal, at step 212. In embodiments, the steady state or updated steady-state phase and amplitude of the PLI are inversed for each segment and added to the digital sample of the composite signal of the corresponding segment. Step 212 outputs the PHSI without the noise or interference (signal 430) from the PLI.

In embodiments, the systems and methods described in the present specification overcome several limitations known in the conventional filtering methods and systems used to remove a mains component from a bio-signal.

First, embodiments of the present specification use the mains signal itself as a reference rather than the component of the mains signal that is found in the composite of the mains signal and the bio-signal. Some embodiments are configured to generate a digital replica of the mains signal using the actual mains signal, which is, in turn, used as the reference.

Second, the reference signal is based on a small number of highly characterized preceding and subsequent samples taken from the actual mains signal and uses a trained computation network to control the synthesizer used to generate the reference signal with instantaneous response to changes in the actual mains signal. Conversely, in the conventionally used systems, a time constant for adaptation to changes in the actual mains signal is approximately 5 to 10 seconds under ideal conditions, and is even slower when low amplitude noise is dominated by ongoing physiological activity and/or noise harmonics are greater than 1 kHz. Therefore, the replica of the mains signal in such systems may lag behind if the mains signal characteristics suddenly change. Under these conditions, some noise/interference due to the mains will transiently appear on the output of the composite signal while the conventional system adapts to the new characteristics. Thus, electrical interference due to the mains cannot be completely cancelled from the bio-signal in the conventional systems.

Third, embodiments of the present specification support removing the mains and the harmonics of the mains signal component in segments. Machine learning computational methods used by the present specification enable dynamic adaptation of the strategy used to remove the mains component for each segment. Additionally, embodiments of the present specification provide the advantage of operating on time sampled data whose sample rate is specified, as opposed to the analog data used by conventional systems and methods.

The above examples are merely illustrative of the many applications of the system of present specification. Although only a few embodiments of the present invention have been described herein, it should be understood that the present invention might be embodied in many other specific forms without departing from the spirit or scope of the invention. Therefore, the present examples and embodiments are to be considered as illustrative and not restrictive, and the invention may be modified within the scope of the appended claims.

What is claimed is:

1. A signal processing apparatus for processing a composite signal comprising a power line interference (PLI) component and a physiological signal of interest (PHSI), wherein the signal processing apparatus comprises:
a hardware component configured to measure an analog PLI signal from a source of the PLI component; and
a processing system configured to:
generate a reference signal by digitizing the measured PLI component;
determine a phase and an amplitude of the measured PLI component using the generated reference signal for at least one segment of the composite signal, wherein each at least one segment comprises a plurality of single cycles;
extract each of the plurality of single cycles from each of the at least one segment from the composite signal and comparing each extracted cycle to the other extracted cycles to identify those signals that have the highest correlation value for the PLI component;
determine, concurrently, a signal to noise ratio (SNR) for each of the at least one segment of the composite signal;
generate a steady state phase and amplitude of the PLI component based on the determined phase and amplitude of the measured PLI component and a weight-adjusted SNR for each of the at least one segment; and
process the composite signal, using at least one of the steady state phase and amplitude of the PLI component or the highest correlation value to remove the PLI component from the composite signal to obtain the PHSI.

2. The apparatus of claim 1, wherein in determining the SNR for each at least one segment, the PLI component is the signal represented by in-phase power and the PHSI component is the noise represented by out-of-phase power.

3. The apparatus of claim 2, wherein a steady state PLI is obtained by measuring the PLI at a time when the PHSI is minimal.

4. The apparatus of claim 3, wherein the processing system is further configured to integrate and filter the PLI component in the composite signal using sequential segments of the composite signal.

5. The apparatus of claim 4, wherein after integration and filtering, the value of each PLI component is weighted according to its dynamically computed SNR.

6. The apparatus of claim 1, wherein the processing system is configured to synchronize the reference signal with a sample rate of the measured PLI.

7. The apparatus of claim 1, wherein the processing system is configured to determine the phase of the measured PLI component using the sine and cosine values of the reference signal.

8. The apparatus of claim 1, wherein the source of the PLI component is a mains power supply.

9. The apparatus of claim 1, wherein the PHSI is a bioelectrical signal.

10. The apparatus of claim 1, wherein the processing system is further configured to:
delay the composite signal in order to detect a change in the PLI component;
recompute the PLI component;
apply the recomputed PLI component retroactively to the composite signal to determine an updated phase and amplitude of the PLI component for each at least one segment, using the PLI component that follows the delayed composite signal; and
subtract the updated phase and amplitude of the PLI from the composite signal to obtain the PHSI.

11. A method for processing a composite signal comprising a PLI component and a PHSI, wherein the method comprises:
measuring a PLI from a source of the PLI component, wherein the PLI is an analog signal, and wherein the PLI contributes to the composite signal in the form of the PLI component;
generating a reference signal by digitizing the PLI;
determining a phase and an amplitude of the PLI component using the generated reference signal, wherein the determining is performed for each segment of a plurality of segments of the composite signal;
determining a signal to noise ratio (SNR) for each segment of the plurality of segments;
generating a steady state phase and amplitude of the PLI component based on the determined phase and amplitude and a weight-adjusted SNR;
processing the composite signal, using the steady state phase and amplitude of the PLI component to removing the PLI component from the composite signal and obtain the PHSI.

12. The method of claim 11, wherein in determining the SNR for each segment, the PLI component is the signal and the PHSI component is the noise.

13. The method of claim 12, wherein a steady state PLI is obtained by measuring the PLI at a time when the PHSI is minimal.

14. The method of claim 13, wherein the PLI component in the composite signal is integrated and filtered using sequential segments of the composite signal.

15. The method of claim 14, wherein after integration and filtering, the value of each PLI component is weighted according to its dynamically computed SNR.

16. The method of claim 11, wherein the reference signal is synchronized with a sample rate of the PLI.

17. The method of claim 11, wherein the reference signal provides sine and cosine values to determine the phase of the PLI component.

18. The method of claim 11, wherein the PLI is a signal generated by a mains power supply.

19. The method of claim 11, wherein the PHSI is a bioelectrical signal.

20. The method of claim 11, further comprising:
delaying the composite signal in order to detect a change in the PLI component;
recomputing the PLI component;
applying the recomputed PLI component retroactively to the composite signal to determine an updated phase and amplitude of the PLI component for each segment, using the PLI component that follows the delayed composite signal; and
subtracting the updated phase and amplitude of the PLI from the composite signal to obtain the PHSI.
